(19) United States
(12) Patent Application Publication
(10) Pub. No.: US 2008/0090859 A1
(43) Pub. Date: Apr. 17, 2008

(54) Use of Selective Opiate Receptor Modulators In the Treatment Of Neuropathy

(75) Inventor: Gerd Bartoszyk, Darmstadt (DE)

Correspondence Address: MORRISON & FOERSTER LLP, 12531 HIGH BLUFF DRIVE, SUITE 100, SAN DIEGO, CA, 92130-2040, US

(73) Assignee: TIOGA PHARMACEUTICALS, INC., San Diego, CA (US)

(21) Appl. No.: 10/577,603

(22) PCT Filed: Oct. 14, 2004
(86) PCT No.: PCT/EP04/11548

§ 371 Date: Oct. 3, 2006

(30) Foreign Application Priority Data
Oct. 30, 2003 (EP) 03024781.1

(51) Int. Cl. A61K 31/439; A61K 31/40; A61K 31/445; A61P 25/00; A61K 31/451; A61K 31/4025

(52) U.S. Cl. 514/295; 514/327; 514/331; 514/409; 514/424

(57) ABSTRACT

The instant invention relates to the use of compounds that are effective as selective opiate receptor modulators for the manufacture of a pharmaceutical for the diagnosis, prophylaxis and/or the treatment of neuropathy, the clinical pictures and symptoms associated therewith, and related disorders, and to pharmaceutical compositions, comprising one or more of said modulator compounds.

The instant invention relates to the use of compounds that are effective as selective opiate receptor modulators for the manufacture of pharmaceuticals for the diagnosis, prophylaxis and/or the treatment of neuropathy and the clinical pictures and symptoms associated therewith.

Neuropathy, or peripheral neuropathy, is a general term referring to disorders of peripheral nerves, usually a nerve damage. The peripheral nervous system is made up of the nerves that branch out of the spinal cord to all parts of the body. Peripheral neuropathy also can be classified by where it occurs in the body. Nerve damage that occurs in one area of the body is called mononeuropathy, in many areas, polyneuropathy. Radiculopathy is the term for neuropathy that affects nerve roots. When the disorder occurs in the same places on both sides of the body, the condition is called symmetric neuropathy. Peripheral neuropathy can be caused by diabetes, vitamin deficiencies, HIV, cancer, viruses, alcohol abuse, or occurs as a side effect of drugs. It therefore can be categorized by cause, such as diabetic neuropathy, nutritional neuropathy or alcoholic neuropathy. When a cause cannot be identified, the condition is called idiopathic neuropathy. Peripheral Neuropathy is common, often distressing, and sometimes disabling. The aetiologie, clinical picture, occurrence and/or interaction with other diseases is extensively discussed in the literature, for example in Boulton, Diabetes Metab 1998; 24 (Suppl 3): 55-65; IIIa, Eur Neurol 1999; 41 (Suppl 1): 3-7; Lagueny, Rev Prat 2000; 50: 731-735; Peltier and Russell, Curr Opin Neurol 2002; 15: 633-638; Simpson, J Neurovirol 2002; 8 (Suppl 2): 3341; Sweeney, Clin J Oncol Nurs 2002; 6:163-166; and Wulff and Simpson, Semin Neurol 1999; 19: 157-164. The disclosure of the cited publications and the references cited therein is explicitly incorporated into this application by reference.

The most common complication of diabetes is neuropathy. It is estimated that up to 60 percent of diabetic patients develop neuropathy in the course of the disease. The neuropathy is typically associated with a wide variety of symptoms, i.a. numbness or tingling sensations, very distressing pins and needles sensation or one similar to receiving a series of electric shocks, and differend kinds and states of pain. Those Symptoms can occur individually or cumulative. If the symptoms of the diabetes-related or diabetes-associated neuropathy include pain, the disease is preferably also referred to as painful diabetic neuropathy or PDN.

The symptoms of diabetic neuropathy and especially the pain associated therewith most often affects the feet and ankles and to a lesser extent the legs above the knees and the arms. Poor control of blood sugar regularly leads to nerve damage, which in turn leads with significant likeliness to the development of the clinical picture of neuropathy. For diabetic neuropathy, the cause of the peripheral neuropathy is known, i.e. diabetes mellitus, and is thought to be related to poor control of blood sugar levels and the resulting hyperglycaemia. The higher the blood sugar level and the longer it remains above the norm the more severe the disease may be. The precise mechanisms that link raised blood sugar to nerve damage still need to be established and other factors, such as abnormalities in nerve growth factors, or the effects of cardiovascular disease (ischaemia, hypoxia) have also been suggested as important contributory factors (for review e.g. Jude and Boulton, Diabetes Reviews 1999; 7: 395410; Dworkin, Clin J Pain 2002; 18: 343-349; Simmons and Feldman, Curr Opin Neurol 2002; 15: 595-603; Barbano et al., Curr Pain Headache Rep 2003; 7: 169-177; Spruce et al., Diabet Med 2003; 20: 88-98).

It was therefore object of the instant invention to make available pharmaceutically active compounds which can be used for the successful diagnosis, prophylaxis and/or the treatment, preferably prophylaxis and/or the treatment, of neuropathy, the clinical pictures and symptoms associated therewith, and related disorders. These pharmaceutically active compounds should provide advantageous properties in comparison to prior art, such as a higher efficiency or potency, improved selectivity and/or little or no adverse effects, especially little or no negative interaction with the central nervous system of the patient treated therewith.

Surprisingly, it was found that compounds that are effective as selective opiate receptor modulators and especially compounds that are effective as peripherally selective opiate receptor modulators can be successfully used in the diagnosis, prophylaxis and/or the treatment of neuropathy, the clinical pictures and symptoms associated therewith, and related disorders.

Furthermore, the compounds that are effective as selective opiate receptor modulators as described herein preferably accelerate nerve regeneration and thus more preferably are able to accelerate or induce the healing of the pathological states or disorders described herein, such as a partial or full reversion of the neuropathy. Additionally, the compounds that are effective as selective opiate receptor modulators as described herein preferably show less adverse effects than the pharmaceuticals of prior art.

Accordingly, subject of the present invention is the use of a compound that is effective as selective opiate receptor modulator for the manufacture of a pharmaceutical for diagnosis, prophylaxis and/or the treatment of neuropathy, the clinical pictures and symptoms associated therewith, and related disorders.

Neuropathies according to the invention are preferably selected from the group consisting of painful diabetic neuropathy, but diabetes induced neuropathies, nutrition induced neuropathies, vitamin deficiency induced neuropathies, HIV induced neuropathies, cancer induced neuropathies, virus induced neuropathies, alcohol abuse induced neuropathies and drug induced neuropathies, more preferably diabetes induced neuropathies, nutrition induced neuropathies and alcohol abuse induced neuropathies and especially diabetes induced neuropathies or diabetic neuropathies. Preferably, neuropathies according to the invention include or consist of painful diabetic neuropathy.

Preferably, said opiate receptor is a kappa-opiate receptor.

Preferably, said receptor modulator is peripherally selective to the receptor.

Preferably, said receptor modulator is a receptor agonist or a receptor antagonist and especially preferred, said receptor modulator is a receptor agonist.

Accordingly, subject of the present invention is the use of a compound that is effective as a peripherally selective kappa-opiate receptor agonist for the manufacture of a pharmaceutical for diagnosis, prophylaxis and/or the treatment of neuropathy, the clinical pictures and symptoms associated therewith, and related disorders.

Compounds that are effective as selective opiate receptor modulators according to the invention also show a high efficacy in neuropathy of other aetiology, and related disorders, clinical pictures or indications, such as post-herpetic neuralgia, chemotherapy induced neuropathy, vulvodynia; and/or lupus erythematosus. The efficiency or potency of compounds that are effective as selective opiate receptor modulators according to the invention in the prophylaxis and/or the treatment of said disorders can be shown according to methods known in the art or in an analogous manner thereof, for example as described in Backonja and Glanzman, Clin Ther 2003; 25: 81-104; Bates and Timmins, Int J STD AIDS 2002; 13: 210-212; Carrazana and Mikoshiba, J Pain Symptom Manage 2003; 25(5 Suppl): S31-35; Harel et al., Pediatr Neurol 2002; 27: 53-56; Jensen, Eur J Pain 2002; 6 (Suppl A): 61-68; LaSpina et al. Eur J Neurol 2001; 8: 71-75; Lersch et al., Clin Colorectal Cancer 2002; 2: 54-58; and/or Mellegers et al., Clin J Pain 2001; 17: 284-295), or in an analogous manner thereof. The disclosure of the cited publications and the references cited therein is explicitly incorporated into this application by reference.

A preferred embodiment of the instant invention therefore relates to the use of a compound that is effective as selective opiate receptor modulator, especially as peripherally selective opiate receptor modulator, for the manufacture of a pharmaceutical for the treatment of disorders selected from group consisting of neuropathy of other aetiology, and related disorders, clinical pictures or indications, and preferably selected from the group consisting of post-herpetic neuralgia, chemotherapy induced neuropathy, vulvodynia; and lupus erythematosus.

Compounds that are effective as selective opiate receptor modulators, especially as peripherally selective opiate receptor modulators, or more precisely, compounds that show selective activity against opiate receptors especially peripheral opiate receptors, are known to the skilled artisan and have been extensively described in the literature. These modulators are commonly divided into opiate receptor agonists and opiate receptor antagonists. Over the years, different subtypes of opiate receptors have been found and studied in detail, the kappa-opiate receptor (or κ-opiate receptor) and the mu-opiate receptor (or μ-opiate receptor) belonging to the most prominent.

Suitable for use according to the invention are compounds that are effective as selective opiate modulators, preferably as peripherally selective opiate modulators, more preferably peripherally selective opiate agonists, even more preferred peripherally selective kappa- or mu-opiate agonists and especially preferred peripherally selective kappa-opiate agonists. These compounds are referred to hereinafter as “compounds for use according to the invention” or as “modulating compounds”.

Various such modulating compounds are known in the art, for example from the subsequent cited literature:

DE-A1-3935371; DE 40 34 785, DE-A4215231; EP-A-0569 802; EP 0 752 246; J. N. Sengupta et al., Pain 79 (1990) 175-185; Laurent Diop et al., European Journal of Pharmacology, 271 (1994) 65-71; Gottschlich et al., Chirality 6: 685-689 (1994); Gottschlich et al., Drugs Exptl. Clin. Res. XXI (5), 171-174 (1995); A. Barber et al., Br. J. Pharmacol. (1994), 113, 1317-1327; and J. N. Junien, P. Riviere, Aliment. Pharmacol. Ther 1995,: 9: 117-126; and the literature cited in the above referenced publications, which are both incorporated into the disclosure of the instant invention by reference.

The modulating compounds disclosed in the above cited references are included into this application by reference. Accordingly, the use of these modulating compounds for the manufacture of a pharmaceutical according to the invention is claimed subject matter of the present invention.

Further compounds for use according to the invention can be readily determined by the skilled artisan, for example by methods known and established in the art or analogously to these established methods, for example by receptor-binding assays, high throughput screening, in vitro testing-systems, in vivo-testing systems, animal models and the like. Examples for methods that can be used to identify compounds for use according to the invention are cited hereinafter:

Krimmer, E. C. et al., Fed. Proc. 1982 (5), 41(7): 2319-22; Spetea et al., Life Sciences 69 (2001), 1775-1782 and Lathi et al., European Journal Pharmacology 1985, 109: 281-284; and the literature cited in the above referenced publications, which are both incorporated into the disclosure of the instant invention by reference.

The activity or potency of a compound for use according to the invention can be determined according to methods known in the art, or an analogous manner thereof. Suitable methods include, but are not limited to, preclinical methods, such as in vitro assays, in vivo assays, cell-based assays and animal models, and clinical methods or clinical studies. Suitable methods are described, for example, in Kim and Chung, Pain. 1992; 50: 355-363; Butelman et al., J Pharmacol Exp Ther 2003; 306: 1106-1114; Field et al., Pain 1999; 80: 391-398; Miki et al., Eur J Pharmacol 2001; 430: 229-234; Wallin et al., Eur J Pain 2002; 6: 261-272; Backonja, Epilepsia 1999; 40 (suppl 6): S57-59; Gorson et al., J Neurol Neurosurg Psychiatry 1999; 66: 251-252; Dallocchio et al., J Pain Symptom Manage 2000; 20: 280-285; Hemstreet and Lapointe, Clin Ther 2001; 23: 520-531; Brooks-Rock, Nurse Pract2001; 26: 59-61; Backonja and Glanzman, Clin Ther2003; 25: 81-104; Kaul et al., Arch Int Pharmacodyn Ther 1978; 234: 139-44; and Calcutt et al., Anesthesiology 2000; 93:1271-1278. The disclosure of the cited publications and the references cited therein is explicitly incorporated into this application by reference.

For example, the streptozotocin diabetic rat model is regarded as a suitable animal model for the study of insulin-deficient diabetes and/or disorders and the corresponding symptoms related thereto, especially the disorders and symptoms as described herein (see, for example, Kaul et al., Arch Int Pharmacodyn Ther 1978; 234: 13944; Calcutt et al., Anesthesiology 2000; 93:1271-1278). In that model, induced short-term diabetes causes sensory disorders in rats ranging from thermal hypoalgesia to exaggerated behavioral responses to other sensory stimuli, and impaired neurotrophic support may promote sensory nerve disorders during diabetes.

The compounds for use according to the invention preferably also show a high efficacy in neuropathy of other aetiology, and related disorders, clinical pictures or indications, such as post-herpetic neuralgia, chemotherapy induced neuropathy, vulvodynia; and/or lupus erythematosus. The efficiency or potency of asimadoline in the prophylaxis and/or the treatment of said disorders can be shown according to methods known in the art or in an analogous manner thereof, for example as described in Backonja and Glanzman, Clin Ther 2003; 25: 81-104; Bates and Timmins, Int J STD AIDS 2002; 13: 210-212; Carrazana and Mikoshiba, J Pain Symptom Manage 2003; 25(5 Suppl): S31-35; Harel et al., Pediatr Neurol 2002; 27: 53-56; Jensen, Eur J Pain 2002; 6 (Suppl A): 61-68; LaSpina et al. Eur J Neurol 2001; 8: 71-75; Lersch et al., Clin Colorectal Cancer 2002; 2: 54-58; and/or Mellegers et al., Clin J Pain 2001; 17: 284-295), or in an analogous manner thereof. The disclosure of the cited publications and the references cited therein is explicitly incorporated into this application by reference.

A preferred embodiment of the instant invention therefore relates to the use of a compound that is effective as selective opiate receptor modulator, more preferred as peripherally selective opiate receptor modulator, even more preferred as peripherally selective kappa-opiate receptor modulator and especially as peripherally selective kappa-opiate receptor agonist, for the manufacture of a pharmaceutical for the treatment of disorders selected from group consisting of neuropathy of other aetiology, and related disorders, clinical pictures or indications, such as post-herpetic neuralgia, chemotherapy induced neuropathy, vulvodynia and/or lupus erythematosus, and preferably selected from the group consisting of post-herpetic neuralgia, chemotherapy induced neuropathy, vulvodynia and/or lupus erythematosus.

In general, compounds are to be regarded suitable as selective opiate receptor modulators for use according to the invention, i. e. modulating compounds, if they show an affinity to one or more opiate receptor, preferably to the mu- and kappa-opiate receptor, more preferably to the mu- or the kappa-opiate receptor and especially to the kappa-opiate receptor that lies, determined as IC₅₀-value, in the range of 100 μmol or below, preferably 10 μmol or below, more preferably in the range of 3 μmol or below, even more preferably in the range of 1 μmol or below and most preferably in the nanomolar range. Especially preferred for use according to the invention are opiate receptor modulators as defined above/below, that are peripherally selective acting opiate receptor modulators. In many cases an IC₅₀-value at the lower end of the given ranges is advantageous and in some cases its highly desirable that the IC₅₀-value is as small as possible, but in general IC₅₀-values that lie between the above given upper limits and a lower limit in the region of 0.0001 μmol 0.001 μmol, 0.01 μmol or even above 0.1 μmol are sufficient to indicate the desired pharmaceutical activity.

The meaning of peripherally selective activity of a compound, preferably of a pharmaceutically active compound or of a pharmaceutical containing such a compound, is known in the art and can be readily determined according to known procedures.

A peripherally selective compound according to the invention preferably means a compound that shows a selectivity for the peripheral nervous system when interacting with the body and preferably with the nervous system of the patient when administered to said patient. Peripherally selective compounds preferably thus show little or even more preferably no detectable impact on the central nervous system of the patient upon administration to said patient.

Preferred compounds for use according to the invention are compounds of formula I

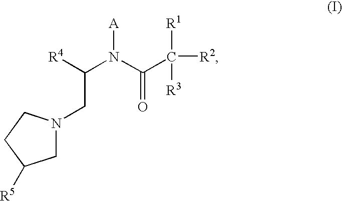

in which
R¹ is Ar, cycloalkyl having 3-7 C atoms or cycloalkylalkyl having 4-8 C atoms,
R² is Ar,
R¹ and R² together are also

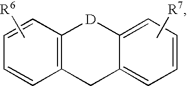

R³ is H, OH, OA or A,
R⁴ is A or phenyl which can optionally be mono- or disubstituted by Hal, OH, OA, CF₃, NO₂, NH₂, NHA, NHCOA, NHSO₂A or NA₂,
R⁵ is OH, CH₂OH,
R⁶ and R⁷ in each case independently of one another are H, Hal, OH, OA, CF₃, NH₂, NHA, NA₂, NHCOA, NHCONH₂, NO₂ or methylenedioxy,
A is alkyl having 1-7 C atoms,
Ar is a mono- or bicyclic aromatic radical which can optionally contain an N, O or S atom and can be mono-, di- or trisubstituted by A, Hal, OH, OA, CF₃, NH₂, NHA, NA₂, NHCOA and/or NHCONH₂,
D is CH₂, O, S, NH, NA, —CH₂—CH₂—, —CH═CH—, —CH₂NH—, —CH₂— NA— or a bond and
Hal is F, Cl, Br or I,
   and/or the salts and/or pharmaceutical acceptable derivatives thereof, and especially compounds of the formula I in which
Ar is phenyl,
R³ is H, and
A is methyl,
   and/or the salts and/or pharmaceutical derivatives thereof, are pharmaceutically active compounds which are very particularly suitable as peripherally selective opiate receptor modulators for use according to the invention. Especially preferred as compound of the formula I is N-methyl-N-[(1S)-1-phenyl-2-((3S)-3-hydroxypyrrolidin-1-yl) ethyl]-2,2-diphenylacetamide (EMD 61753) and/or a salt and/or a pharmaceutical derivative thereof, preferably a pharmaceutical acceptable salt and especially the hydrochloride salt. This compound is known as Asimadoline.

Other preferred modulating compounds for use according to the invention are selected from a group consisting of Alvimopan (see for example Am. J. Surg. 2001 Nov;182(5A Suppl):27S-38S), Loperamide (see for example J Pharmacol Exp Ther 1999 Apr;289(1):494-502), Spiradoline (see for example Pol. J. Pharmacol. 1994 Jan-Apr;46(1-2):37-41), Fedotozine (see for example Expert Opin Investig Drugs. 2001 January;10(1):97-110), Pentazocine (see for example Biol Pharm Bull. 1997 November;20(11):1193-8), ICI204448 (see for example Br J Pharmacol. 1992 August;106(4):783-9), U-50488H (see for example Life Sci. 2002 March 1;70(15):1727-40), ADL 10-0101 (see for example Pain 2002 March;96(1-2):13-22), ADL 10-0116 (see for example Pain 2002 March;96(1-2):13-22) and ADL 1-0398 (from Adolor Corp., USA)

In one preferred embodiment of the invention the modulating compounds are selected from a group consisting of Alvimopan, Loperamide, Fedotazine and Asimadoline.

In another preferred embodiment of the invention the modulating compounds are selected from a group consisting IC1204448, U-50488H, ADL 10-0101, ADL 10-0116 and ADL 1-0398.

In a more preferred embodiment of the invention, the modulating compounds are selected from a group consisting of Alvimopan, Loperamide, Asimadoline, ADL 10-0116 and ADL 1-0398.

Especially preferred for use according to the invention is Asimadoline or a salt or solvate thereof.

According to the invention, the term “pharmaceutical for the diagnosis of disorders” comprises pharmaceuticals that are used directly for diagnostic purposes as well as pharmaceuticals that enable or facilitate the application of diagnostic methods, for example by influencing the sensitivity, especially the sensitivity to pressure and pain in the patient. Moreover, the modulating compounds can advantageously applied directly as diagnostic tool, for example for distinguishing the disorders as described herein from other disorders and/or to determine the respective aetiology of the disorder and especially to determine the subtypes of the disorders (for example by the dependency of the disorder from the subtypes of opiate receptors) as described herein, which can be of extraordinary therapeutic value.

The compounds for use according to the invention are additionally advantageous as they preferably do not pass the blood-brain barrier or only to a minor, not relevant extent. This minimizes the risks of unwanted adverse effects.

Furthermore the compounds for use according to invention do not, or only to a minor, not relevant extent, interact with the Central nervous system of the patient they are administered to.

Compounds for use according to the present invention are preferably selected from compounds which cannot pass through the blood-brain barrier on account of their structure and therefore do not exhibit a dependence potential. Also, until now no actions have been found which would restrict the use of the advantageous actions for the claimed indications in any way.

In all indication areas, clinical pictures or symptoms described here, in particular the use of N-methyl-N-[(1S)-1-phenyl-2-((3S)-3-hydroxypyrrolidin-1-yl)ethyl]-2,2-diphenylacetamide and especially the use of N-methyl-N-[(1S)-1-phenyl-2-((3S)-3-hydroxypyrrolidin-1-yl)ethyl]-2,2-diphenylacetamide, hydrochloride (Asimadoline) as modulating compound and thus as a pharmaceutical or as active ingredient in a pharmaceutical has emerged as particularly effective. This particular high efficiency of asimadoline in all indications described herein is preferably maintained in all sorts of preparation forms.

Asimadoline preferably shows a high efficiency or potency in the prophylaxis and/or the treatment of neuropathy and/or the symptoms associated therewith.

More preferably, Asimadoline shows a high efficiency or potency in the prophylaxis and/or the treatment of diabetic neuropathy and/or the symptoms associated therewith.

Accordingly, a preferred subject of the invention relates to the use of N-methyl-N-[(1S)-1-phenyl-2-((3S)-3-hydroxypyrrolidin-1-yl)ethyl]-2,2-diphenyl-acetamide and/or the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof, for the manufacture of a pharmaceutical for the diagnosis, prophylaxis and/or treatment of neuropathy, especially diabetic neuropathy.

Accordingly, an especially preferred subject of the invention relates to the use of N-methyl-N-[(1S)-1-phenyl-2-((3S)-3-hydroxypyrrolidin-1-yl)ethyl]-2,2-diphenylacetamide and especially the use of N-methyl-N-[(1S)-1-phenyl-2-((3S)-3-hydroxypyrrolidin-1-yl)ethyl]-2,2-diphenylacetamide, hydrochloride, for the manufacture of a pharmaceutical for the diagnosis, prophylaxis and/or treatment of neuropathy, especially diabetic neuropathy.

A further preferred embodiment of the instant invention relates to the use of N-methyl-N-[(1S)-1-phenyl-2-((3S)-3-hydroxypyrrolidin-1-yl)ethyl]-2,2-diphenylacetamide and especially the use of N-methyl-N-[(1S)-1-phenyl-2-((3S)-3-hydroxypyrrolidin-1-yl)ethyl]-2,2-diphenylacetamide, and/or the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof, for the manufacture of a pharmaceutical for the diagnosis, prophylaxis and/or treatment of disorders, clinical pictures or indications, selected from the group consisting of post-herpetic neuralgia, chemotherapy induced neuropathy, vulvodynia and lupus erythematosus.

A further especially preferred embodiment of the instant invention relates to the use of of N-methyl-N-[(1S)-1-phenyl-2-((3S)-3-hydroxypyrrolidin-1-yl)ethyl]-2,2-diphenylacetamide and especially the use of N-methyl-N-[(1S)-1-phenyl-2-((3S)-3-hydroxypyrrolidin-1-yl)ethyl]-2,2-diphenylacetamide, hydrochloride, for the manufacture of a pharmaceutical for the diagnosis, prophylaxis and/or treatment of disorders, clinical pictures or indications, selected from the group consisting of post-herpetic neuralgia, chemotherapy induced neuropathy, vulvodynia and lupus erythematosus.

A further especially preferred embodiment of the instant invention relates to the use of of N-methyl-N-[(1S)-1-phenyl-2-((3S)-3-hydroxypyrrolidin-1-yl)ethyl]-2,2-diphenylacetamide and especially the use of N-methyl-N-[(1S)-1-phenyl-2-((3S)-3-hydroxypyrrolidin-1-yl)ethyl]-2,2-diphenylacetamide, hydrochloride, for the manufacture of a pharmaceutical for the diagnosis, prophylaxis and/or treatment of disorders, selected from the group consisting of post-herpetic neuralgia, vulvodynia, lupus erythematosus and chemotherapy induced neuropathy.

An especially preferred embodiment of the instant invention relates to the use of the compound N-methyl-N-[(1S)-1-phenyl-2-((3S)-3-hydroxypyrrolidin-1-yl)ethyl]-2,2-diphenylacetamide, hydrochloride, for the manufacture of a pharmaceutical for the prophylaxis and/or treatment of neuropathy, especially diabetic neuropathy.

According to the invention, the use of the modulating compounds for the manufacture of a pharmaceutical for the prophylaxis and/or treatment of the disorders, clinical pictures and/or symptoms as described herein is preferred.

Further subject of the instant invention is the use of the modulating compounds as defined herein in the diagnosis, prophylaxis and/or treatment, preferably prophylaxis and/or treatment, of disorders, clinical pictures and/or symptoms as described herein.

Further preferred subject of the invention is the use of the modulating compounds as defined herein in the diagnosis, prophylaxis and/or treatment, preferably prophylaxis and/or treatment of neuropathy, the clinical pictures and symptoms associated therewith, and related disorders as described herein.

According to the invention, the use of the modulating compounds for the manufacture of a pharmaceutical for the treatment the disorders, clinical pictures and/or symptoms as described herein is especially preferred.

The compounds for use according to the present invention and/or their physiologically acceptable salts and/or their physiologically acceptable derivatives can therefore be used for the production of pharmaceutical compositions or preparations by bringing them into the suitable dose form together with at least one excipient or auxiliary and, if desired, with one or more further active compounds. The compositions or preparations thus obtained can be employed as medicaments in human or veterinary medicine. Suitable excipients are organic or inorganic substances which are suitable for enteral (e.g. oral or rectal) or parenteral administration and do not react with the compounds for use according to the present invention, for example water, vegetable oils, benzyl alcohols, polyethylene glycols, glycerol triacetate and other fatty acid glycerides, gelatin, soya lecithin, carbohydrates such as lactose or starch, magnesium stearate, talc or cellulose.

For oral administration, in particular tablets, coated tablets, capsules, syrups, juices or drops are used. Of interest are especially coated tablets and capsules having enteric coatings or capsule shells. For rectal administration, suppositories are used, and for parenteral administration, solutions, preferably oily or aqueous solutions, and also suspensions, emulsions or implants are used.

The compounds for use according to the invention can also be lyophilized and the lyophilisates obtained used, for example, for the production of injection preparations.

The compositions or preparations indicated can be sterilized and/or contain auxiliaries such as preservatives, stabilizers and/or wetting agents, emulsifiers, salts for affecting the osmotic pressure, buffer substances, colourants and/or flavourings. If desired, they can also contain one or more further active compounds, e.g. one or more vitamins, diuretics, anti-inflammatorial compounds, anti-diabetics, analgetics, anti-phlogistics or compounds other than the compounds for use according to the invention, such as compounds that are not subject of the present invention, that can be used in the diagnosis, prophylaxis and/or treatment of the disorders, clinical pictures and/or symptoms as described herein, for example to improve or enhance the therapeutic effect and/or tolerance of the modulating compounds further.

Further subject of the invention is a pharmaceutical composition, characterized in that it comprises a pharmaceutically effective amount of one or more compounds effective as selective opiate receptor modulators as defined herein (modulating compounds).

Further subject of the invention is a pharmaceutical composition, characterized in that it comprises a pharmaceutically effective amount of two or more compounds effective as selective opiate receptor modulators as defined herein (modulating compounds).

Further subject of the invention is a pharmaceutical composition as described above/below, characterised in that it contains one or more additional compounds, selected from the group consisting of physiologically acceptable excipients, auxiliaries, adjuvants, carriers and pharmaceutically active ingredients other than the modulating compounds according to the invention.

Preferably, the pharmaceutically active ingredients other than the modulating compounds are selected from anti-diabetics and pharmaceutically active ingredients (other than the modulating compounds) useful for the diagnosis, prophylaxis and/or the treatment of neuropathy, the clinical pictures and symptoms associated therewith.

More preferably, the pharmaceutically active ingredients (other than the modulating compounds) useful for the diagnosis, prophylaxis and/or the treatment of neuropathy, the clinical pictures and symptoms associated therewith are selected from pharmaceutically active ingredients for the symptomatic treatment of the disorders, clinical pictures and/or symptoms as described herein, such as alpha-lipoic acid, neurotropic vitamins, especially vitamin B₆ and vitamin B₁₂, anticonvulsants, especially Gabapentin, calcium-antagonists, Baclofen, Diclofenac, Metamizol, chinin, local anesthetics, analgetics, especially central acting analgetics, and antidepressants, especially tricyclic antidepressants, and combinations thereof. More preferably, they are selected from alpha-lipoic acid, vitamin B₆, vitamin B,₁₂, Gabapentin, Baclofen, Diclofenac, Metamizol, chinin and the combination of Metamizol and chinin, and combinations thereof.

According to the invention, modulating compounds as defined herein are not pharmaceutically active ingredients for the symptomatic treatment of the disorders, clinical pictures and/or symptoms as described herein.

Thus, a preferred embodiment of the instant invention relates to the combination of one or more of the modulating compounds according to the invention and one or more compounds other than the modulating compounds according to the invention that are selected from anti-diabetic drugs or anti-diabetics and combinations thereof.

Accordingly, subject of the present invention is a pharmaceutical composition that comprises a pharmaceutically effective amount of one or more modulating compounds, one or more anti-diabetic drugs or anti-diabetics and combinations thereof, and optionally one or more compounds, selected from the group consisting of physiologically acceptable excipients, auxiliaries, adjuvants, carriers and pharmaceutically active ingredients other than the modulating compounds according to the invention.

Anti-diabetics according to the invention include, but are not limited to, Insulines, such as naturally derived, conventionally derived or gene technology derived Insulines, preferably selected from short-acting Insulines, rapid-acting Insulines, retarded Insulines, long-acting Insulines and combination products comprising Insulines and in all application forms thereof, such as oral, parenteral, per injectionem, nasal and pulmonal; α-Glucosidase-Inhibitors, such as Acarbose, Miglitol and Voglibose; Biguanides, such as Metformin; Sulfonyl ureas, such as Carbutamid, Tolbutamid, Glibornurid, Glibenclamid, Glimepirid, Gliquidon, Glisoxepid, Gliclazid, Glisentid, Glipizid, Glisolamid, Chlorpropamid and Glyburid; Insulin-Sensitizer, especially Thiazolidendione and Glitazone, such as Pioglitazon, Rosiglitazon, Diab II, Isaglitazone, GW-409544, Balaglitazon and rhIGF-1/rhIGFBP-3 complex; Insulinotropin-Antagonists, such as Repaglinide, Glimepiride and Nateglinide; Glucagon-like Peptide 1-Agonists, such as Exenatide, Exenatide LAR and Liraglutide; Calcium channel-Antagonists, such as Mitiglinide; Sodium/glucose cotransporter inhibitors, such as T-1095; Glucagon-Agonists, such as Glucagon; Amylin-Agonists, such as Pramlintid; Monoclonal Antibodies and Derivatives thereof, such as AGT-1 and Daclizumab; Glutamatdecarboxylase Stimulators, such as Diamyd; and TNFα-Antagonists, such as Humicade and Etanercept.

α-Glucosidase-Inhibitors, Biguanides, Sodium/glucose cotransporter inhibitors, Insulinotrope Anti-diabetics, especially Sulfonyl ureas, Insulin-Sensitizer and Insulinotropin-Antagonists, Glucagon-like Peptide 1-Agonists, Calcium channel-Antagonists and Sodium/glucose cotransporter inhibitors are commonly characterised as oral anti-diabetics. Oral anti-diabetics are preferred anti-diabetics in respect to the instant invention.

Suitable anti-diabetics, categories and ways of classification as well as mechanism of action, activity profiles and adverse effects are described in Mutschler, Arzneimittelwirkungen, 8th edition; Mutschler, Forth: Pharmakologie und Toxikologie 2001; Diabetes mellitus—neue Therapien und Strategien—special edition: Forschung und Praxis 2001.

More preferred, anti-diabetics according to the invention are selected from the group consisting of Chlorpropamide, Glibenclamide, Tolbutamide, Metformin, Nateglinide, Repaglinide, Gliclazide, Glipizide, Glimepiride, Pioglitazone and Rosiglitzone.

Further subject of the invention is a Kit consisting of separate packs of
a) a pharmaceutically effective amount of one or more selective opiate receptor modulators as defined herein (modulating compounds) and
b) a pharmaceutically effective amount of one or more compounds, selected from pharmaceutically active ingredients other than the modulating compounds, preferably pharmaceutically active ingredients, other than the modulating compounds, that are suitable for the diagnosis, prophylaxis and/or treatment of neuropathy, the clinical pictures and symptoms associated therewith (other than the modulating compounds), and anti-diabetics.

The kit comprises suitable packs or containers, such as boxes, individual bottles, blister packings, bags or ampoules. The kit may, for example, comprise separate ampoules in each of which there is an effective amount of the respective pharmaceutically active ingredient or ingredients, for example in solid form, dissolved form or lyophylized form. Alternatively, the kit may, for example, comprise separate strips of blistered tablets containing the respective pharmaceutically active ingredient or ingredients, or separate boxes containing the respective strips of blistered tablets.

Preferred is a Kit as described above, wherein separate pack (b) comprises one or more anti-diabetics, preferably anti-diabetics as described above, as the one or more compounds other than the modulating compounds. Optionally, separate pack (b) comprises one or more additional compounds, selected from the group consisting of physiologically acceptable excipients, auxiliaries, adjuvants and carriers.

Preferably, in the separate packs (a) and/or (b), the compounds comprised are comprised as pharmaceutical compositions, respectively, wherein the respective pharmaceutical compositions preferably are as described above/below.

If the compound for use according to the invention is a compound with basic properties, it is usually called a base or free base of the compound. It can be advantageous to convert the free base into the associated acid-addition salt using an acid, for example by reaction of equivalent amounts of the base and the acid in an inert solvent, such as ethanol, followed by evaporation. The Suitable acids for this reaction are, in particular, those which give physiologically acceptable salts. Thus, it is possible to use inorganic acids, for example sulfuric acid, sulfurous acid, dithionic acid, nitric acid, hydrohalic acids, such as hydrochloric acid or hydrobromic acid, phosphoric acids, such as, for example, orthophosphoric acid, sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, hexanoic acid, octanoic acid, decanoic acid, hexadecanoic acid, octadecanoic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, benzenesulfonic acid, trimethoxybenzoic acid, adamantanecarboxylic acid, p-toluenesulfonic acid, glycolic acid, embonic acid, chlorophenoxyacetic acid, aspartic acid, glutamic acid, proline, glyoxylic acid, palmitic acid, parachlorophenoxyisobutyric acid, cyclohexanecarboxylic acid, glucose 1-phosphate, naphthalenemono- and -disulfonic acids or laurylsulfuric acid. Salts with physiologically unacceptable acids, for example picrates, can be used to isolate and/or purify the compounds of the formula I. On the other hand, compounds of the formula I can be converted into the corresponding metal salts, in particular alkali metal salts or alkaline earth metal salts, or into the corresponding ammonium salts, using bases (for example sodium hydroxide, potassium hydroxide, sodium carbonate or potassium carbonate). Suitable salts are furthermore substituted ammonium salts, for example the dimethyl-, diethyl- and diisopropylammonium salts, monoethanol-, diethanol- and diisopropanolammonium salts, cyclohexyl- and dicyclohexylammonium salts, dibenzylethylenediammonium salts, furthermore, for example, salts with arginine or lysine.

Alternatively, compounds for use according to the invention with acidic properties can be converted into the associated base-addition salt using a base, for example by reaction of equivalent amounts of the acidic compound and the base in an inert solvent, such as ethanol, followed by evaporation. Examples for suitable bases are physiologically acceptable amines, hydroxides or carbonates, such as ethanol amine, sodium hydroxide, potassium hydroxide, sodium carbonate and potassium carbonate—oder Kaliumhydroxid oder—carbonat), that transfer the compounds for use according to the invention into the respective ammonium salts or metal salts.

On the other hand, if desired, the free bases of the formula I can be liberated from their salts using bases (for example sodium hydroxide, potassium hydroxide, sodium carbonate or potassium carbonate).

Pharmaceutically acceptable derivatives of compounds for use according comprise prodrugs, metabolites and the like. Examples for such prodrugs and/or metabolites comprise compounds for use according to the invention that are modified with groups that are readily degraded/removed, such as alkyl groups, acyl groups and/or biodegradable polymers, and therefore liberate the compound for use according to the invention from the respective derivative. Examples for suitable biopolymers are described in the literature, for example Int. J. Pharm. 115, 61-67 (1995).

As used herein, the term “solvate” preferably refers to a complex of variable stoichiometry formed by a solute (in this invention, a compound of formula I or a salt or physiologically functional derivative thereof and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water. Examples for suitable solvates are the mono- or dihydrates or alcoholates of the modulating compounds.

The invention furthermore relates to a pharmaceutical composition, comprising one or more compounds effective as a selective opiate receptor modulator as defined above, preferably a pharmaceutical composition, comprising one or more compounds effective as a selective opiate receptor modulator as defined above, characterized in that the compound or the compounds are comprised in pharmaceutically active amounts.

Pharmaceutical compositions according to the invention can be obtained or produced according to methods known in the art or analogously to these methods. Usually, the pharmaceutical compositions according to the invention are produced with non-chemical methods, for example by mixing the active ingredients, i. e. one or more modulating compounds (or a salts thereof) and optionally one or more compounds other than the modulating compounds according to the invention (or a salt thereof), and optionally further ingredients, e. g. physiologically acceptable excipients, auxiliaries, adjuvants and carriers, and converting the mixture into the desired dosage form, for example into tablets by molding methods or into solutions by solving the active ingredients in a solvent. In general, the active ingredients are converted into a pharmaceutical composition together with one or more excipient, for example a solid, liquid and/or semiliquid excipient, or one or more auxiliaries and, if desired, in combination with one or more further active ingredients.

These preparations can be used as medicaments in human or veterinary medicine. Suitable excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatine, carbohydrates, such as lactose or starch, magnesium stearate, talc or vaseline. Suitable for oral administration are, in particular, tablets, pills, coated tablets, capsules, powders, granules, syrups, juices or drops, suitable for rectal administration are suppositories, suitable for parenteral administration are solutions, preferably oily or aqueous solutions, furthermore suspensions, emulsions or implants, and suitable for topical application are ointments, creams or powders. The novel compounds can also be lyophilized and the resultant lyophilizates used, for example, for the preparation of injection preparations. The preparations indicated may be sterilized and/or comprise assistants, such as lubricants, preservatives, stabilizers and/or wetting agents, emulsifiers, salts for modifying the osmotic pressure, buffer substances, dyes, flavours and/or a plurality of further active ingredients, for example one or more vitamins.

For administration as an inhalation spray, it is possible to use sprays in which the active ingredient is either dissolved or suspended in a propellant gas or propellant gas mixture (for example CO₂ or chlorofluorocarbons). The active ingredient is advantageously used here in micronized form, in which case one or more additional physiologically acceptable solvents may be present, for example ethanol. Inhalation solutions can be administered with the aid of conventional inhalers.

As a rule, the modulating compounds according to the invention can generally administered in analogy to other known active ingredients or preparations of prior art, e.g. the ones commercially available, for the indications claimed. However, due to the advantageous properties of the modulating compounds according to the invention, an administration at the lower end of the dosages given for the compounds of prior art is often preferred.

Accordingly, the modulating compounds are preferably administered in doses between about 0.001 mg and 200 mg, more preferably between about 0.01 mg and 100 mg, even more preferably between about 0.01 mg and 50 mg and in particular between 0.01 and 30 mg, per dose unit. Preferred examples of suitable administration doses are selected from about 0.1 mg, about 0.5 mg, about 1.0 mg, about 2.0 mg and about 5.0 mg. Said administration doses (dose units) are preferably administered from once a day up to five times a day, more preferably from once a day up to three times a day. Even more preferably, said administration doses (dose units) are administered once a day or twice a day (BID—Bis In Die).

The daily dose is preferably more than or equal to about 0.0001 mg/kg, more preferably more than or equal to about 0.001 mg/kg, even more preferably more than or equal to about 0.005mg/kg, more than or equal to about 0.01 mg/kg or more than or equal to about 0.1 mg/kg of body weight. The daily dose is preferably less than or equal to about 30 mg/kg, more preferably less than or equal to about 20 mg/kg, even more preferably less than or equal to about 15 mg/kg, less than or equal to about 5 mg/kg or less than or equal to about 1 mg/kg of body weight.

Accordingly, the daily dose is preferably between about 0.0001 and 30 mg/kg, more preferred between about 0.001 and 20 mg/kg, even more preferred between about 0.005 and 15 mg/kg, especially preferred between about 0.01 and 10 mg/kg and in particular between about 0.01 and 2.0 mg/kg of body weight, for example a daily dose selected from about 0.0075 mg/kg of body weight, about 0.0125 mg/kg of body weight, about 0.025 mg/kg of body weight, about 0.075 mg/kg of body weight, about 0.15 mg/kg and about 0.25 mg/kg of body weight. In some cases, it is advantageous if the daily dosis is given in one single dosis. In many cases, it is advantageous if the daily dosis is given in two separate portions each comprising the half amount of the given daily dosis.

In general, notes on the dosage of the modulating compounds in mg are based on the pharmaceutical effective compounds itself or, if the compound is administered as salt, for example as hydrochloride, preferably on the weight of the compound as its salt. The dosage given in mg/kg is based on the body weight of the patient in kg to which the compound is administered.

The specific dose for each individual patient depends, however, on various factors, for example on the activity of the specific compound employed, on the age, body weight, general state of health and sex, on the diet, on the time and route of administration, and on the excretion rate, pharmaceutical combination and severity of the particular disorder to which the therapy applies. Oral administration is preferred.

Subject of treatment or administration according to the aspects of the invention is every patient in need of such a treatment or an administration, preferably an animal, especially and nonhuman mammalian, and especially preferred a human being.

Experimental:

A) Insulin deficient diabetes was induced in rats by a single injection of streptozotocin (50 mg/kg, intraperitoneally). After having confirmed the development of diabetes by established hyperglycemia, determined in blood samples taken from the rats, asimadoline was administered subcutaneously at doses of 1 mg/kg, 5 mg/kg and 15 mg/kg. For comparison, gabapentin, the most recent treatment for diabetic neuropathy, at 50 mg/kg was included. To determine mechanical threshold (tactile allodynia) for foot withdrawal, a series of Von Frey filaments (minimum 0.25 g; maximum 15 g) was applied in sequence to the plantar surface of the right hindpaw with a pressure that causes the filament to buckle, starting with a filament of a buckling weight of 2 g. Absence of a response after 5 seconds was regarded a negative response, and the next heavier filament was applied. Lifting of the paw was recorded as a positive response, and the next lightest filament were applied. This sequence was continued until four measurements have been made after an initial change in the behavior or until five consecutive negative (given the score of 15 g) or four positive (score of 0.25 g) scores were obtained. The resulting sequence of positive and negative scores was used to determine the 50% response threshold.

When measured 3 h after administration, asimadoline dose-dependently inhibited and at the highest dose nearly completely abolished tactile allodynia to level of non-diabetic rats, comparable to gabapentin 50 mg/kg.

The data are shown in table 1. That the beneficial effects are indeed due to the opioid mechanism of asimadoline can also be seen in Table 1. The anti-allodynic effect of the highest dose of asimadoline was completely abolished by the intraplantar (i.pl.) injection of the selective kappa antagonist nor-BNl (nor-binaltorphimine).

TABLE 1

| | | 50% Response Threshold |
|---|---|---|
| Treatment Condition | | Mean ± SEM [g] |
| Non-Diabetic Control | | 11 ± 1.4 |
| Diabetic Control | | 3 ± 0.5 |
| Diabetic + Asimadoline | 1 mg/kg s.c. | 2 ± 0.2 |
| Diabetic + Asimadoline | 5 mg/kg s.c. | 6 ± 2.0 |
| Diabetic + Asimadoline | 15 mg/kg s.c. | 9 ± 1.7 |
| Diabetic + Gabapentin | 50 mg/kg s.c. | 10 ± 1.9 |

| | |
|---|---|
| Non-Diabetic Control + nor-BNI 100 μg i.pl. | 13 ± 1.6 |
| Diabetic + nor-BNI 100 μg i.pl. + | 3 ± 1.0 |
| Asimadoline 15 mg/kg s.c. | |

B) Neuropathic pain was induced by surgical sympathectomy, i.e. the L5 and L6 spinal nerves on one side were tightly ligated in anesthetized rats. After 15 days of recovery, a series of Von Frey filaments (0.4- 15 g) was applied in sequence to the plantar surface of the ligated hind paw to determine the 50% mechanical threshold for foot withdrawal as described before.

When measured 2 h after administration, asimadoline dose-dependently and at the highest dose nearly completely abolished tactile allodynia to the level of non-ligated rats, comparable to gabapentin 100 mg/kg. The data are shown in table 2.

TABLE 2

| | | 50% Response |
|---|---|---|
| | | Threshold |
| Treatment Condition | | Mean ± SEM [g] |
| Non-Ligated Control | | 13.7 ± 0.8 |
| Ligated Control | | 3.0 ± 1.9 |
| Ligated + Asimadoline | 1 mg/kg p.o. | 6.4 ± 0.5 |
| Ligated + Asimadoline | 3 mg/kg p.o. | 10.1 ± 0.6 |
| Ligated + Asimadoline | 10 mg/kg p.o. | 12.0 ± 0.4 |
| Ligated + Gabapentin | 100 mg/kg p.o. | 12.8 ± 1.6 |

C) Because neuropatic pain is characterized by allodynia for various sensory modalities, also thermal allodynia was investigated in a monkey model where allodynia was induced by the topical application of 0.004 Mol of capsaicin to the surface of the tail. Allodynia was assessed by the latency for withdrawal of the tail following immersion of the tail into water with non-noxious temperatures of either 38° C. or 42° C. (cut-off latency of 20 sec).

When measured 45 min after administration, asimadoline dose-dependently and at the highest dose nearly completely abolished thermal allodynia to the level of non-ligated rats under the 38° C. condition. The data are shown in table 3.

TABLE 3

| | | Tail Withdrawal Latency | |
|---|---|---|---|
| | | Mean ± SEM [sec] | |
| Treatment Condition | | 38° C. | 42° C. |
| Non-Capsaicin Control | | 20.0 ± 0.0 | 20.0 ± 0.0 |
| Capsaicin Control | | 3.4 ± 1.5 | 1.1 ± 01. |
| Capsaicin + Asimadoline | 0.01 mg/kg s.c. | 8.0 ± 4.5 | 4.5 ± 2.5 |
| Capsaicin + Asimadoline | 0.032 mg/kg s.c. | 19.6 ± 0.4 | 12.8 ± 4.2 |

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. The use of a compound that is effective as selective opiate receptor modulator for the manufacture of a pharmaceutical for the diagnosis, prophylaxis and/or the treatment of neuropathy, the clinical pictures and symptoms associated therewith, and related disorders.

15. The use according to claim 14, characterized in that said receptor modulator is a receptor agonist.

16. The use according to claim 14, characterized in that said receptor modulator is peripherally selective to the receptor.

17. The use according to claim 14, characterized in that said opiate receptor is a kappa-opiate receptor.

18. The use according to claim 14, characterized in that said receptor modulator is a receptor agonist, that said receptor modulator is peripherally selective to the receptor and that said opiate receptor is a kappa-opiate receptor

19. The use according to claim 14, characterized in that the compound is selected from group consisting of Alvimopan, Loperamide, Asimadoline, Fedotozine, Pentazocine, U62066E, ICI204448, U-50488H, ADL 10-0101, ADL 10-0116 and ADL 1-0398.

20. The use according to claim 14, characterized in that the compound that is effective as selective opiate receptor modulator is N-methyl-N-[(1S)-1-phenyl-2-((3S)-3-hydroxypyrrolidin-1-yl) ethyl]-2,2-diphenylacetamide, and/or a pharmaceutically acceptable derivative, solvate, salt or stereoisomer thereof, including mixtures thereof in all ratios.

21. The use according to claim 14, characterized in that the related disorders are selected from the group consisting of post-herpetic neuralgia, vulvodynia, lupus erythematosus and chemotherapy induced neuropathy.

22. The use according to claim 19, characterized in that the related disorders are selected from the group consisting of post-herpetic neuralgia, vulvodynia, lupus erythematosus and chemotherapy induced neuropathy.

23. The use according to claim 14, characterized in that the compound that is effective as selective opiate receptor modulator is N-methyl-N-[(1S)-1-phenyl-2-((3S)-3-hydroxypyrrolidin-1-yl) ethyl]-2,2-diphenylacetamide, hydrochloride, and that the disorders are selected from the prophylaxis and the treatment of neuropathy.

24. A pharmaceutical composition, characterized in that it comprises a pharmaceutically effective amount of one or more compounds effective as selective opiate receptor modulators as defined in claim 14.

25. A pharmaceutical composition, characterized in that it comprises a pharmaceutically effective amount of one or more compounds effective as selective opiate receptor modulators as defined in claim 19.

26. A pharmaceutical composition, characterized in that it comprises a pharmaceutically effective amount of one or more compounds effective as selective opiate receptor modulators as defined in claim 14, and in that it contains one or more additional compounds, selected from the group consisting of physiologically acceptable excipients, auxiliaries, adjuvants, carriers and pharmaceutically active ingredients other than the compounds according to claim 14.

27. A pharmaceutical composition, comprising a pharmaceutically effective amount of one or more compounds effective as selective opiate receptor modulators as defined in claim 14, further comprising one or more additional compounds, selected from the group consisting of physiologically acceptable excipients, auxiliaries, adjuvants, carriers and pharmaceutically active ingredients other than the compounds according to claim 14, wherein the pharmaceutically active ingredients other than the compounds according to claim 14 are selected from anti-diabetics and pharmaceutically active ingredients other than the compounds according to claim 14 that are useful for the diagnosis, prophylaxis and/or the treatment of neuropathy, the clinical pictures and symptoms associated therewith, and related disorders.

28. A pharmaceutical composition, comprising a pharmaceutically effective amount of one or more compounds according to claim 19, further comprising one or more additional compounds, selected from the group consisting of physiologically acceptable excipients, auxiliaries, adjuvants, carriers and pharmaceutically active ingredients other than the compounds according to claim 19, wherein the pharmaceutically active ingredients other than the compounds according to claim 19 are selected from anti-diabetics and pharmaceutically active ingredients useful for the diagnosis, prophylaxis and/or the treatment of neuropathy, the clinical pictures and symptoms associated therewith, and related disorders, other than the compounds according to claim 19.

29. A method for the manufacture of a pharmaceutical composition according to 26, characterized in that the one or more compounds effective as selective opiate receptor modulator and optionally the one or more additional compounds are mixed together and converted into a pharmaceutical composition suitable for administration.

30. A method for the diagnosis, prophylaxis and/or the treatment of neuropathy, the clinical pictures and symptoms associated therewith, and related disorders, comprising administering to the patient in need thereof a pharmaceutically effective amount of at least one compound that is effective as selective opiate receptor modulator.

31. A method for the diagnosis, prophylaxis and/or the treatment of neuropathy, the clinical pictures and symptoms associated therewith, and related disorders, comprising administering to the patient in need thereof a pharmaceutically effective amount of at least one compound according to claim 19.

32. Kit consisting of separate packs of
a) a pharmaceutically effective amount of one or more selective opiate receptor modulators as defined in claim 14, and
b) a pharmaceutically effective amount of one or more compounds, selected from pharmaceutically active ingredients other than the compounds according to claim 14.

33. Kit consisting of separate packs of
a) a pharmaceutically effective amount of one or more selective opiate receptor modulators as defined in claim 19, and
b) a pharmaceutically effective amount of one or more compounds, selected from pharmaceutically active ingredients other than the compounds according to claim 19.
